(19) United States
(12) Patent Application Publication
(10) Pub. No.: US 2009/0247907 A1
(43) Pub. Date: Oct. 1, 2009

(54) ANATOMIC MEASUREMENT DEVICE, METHOD, AND DOCUMENTATION MEANS

(75) Inventor: Joel Chechik, Minneapolis, MN (US)

Correspondence Address: COLOPLAST A/S;Attention: Corporate Patents, Holtedam 1, DK-3050 Humlebaek, DK

(73) Assignee: Coloplast A/S, Humlebaek (DK)

(21) Appl. No.: 12/416,168

(22) Filed: Apr. 1, 2009

(30) Foreign Application Priority Data
Apr. 1, 2008 (DK) PA 2008 00435

(51) Int. Cl. A61B 5/103

(52) U.S. Cl. 600/587

(57) ABSTRACT

An anatomic measurement device comprises a base sheet having a central longitudinal axis, a proximal base edge, a left side angle axis, and a right side angle axis. Grid markings are provided on the base sheet, corresponding to the central longitudinal axis, the left side angle axis, and the right side angle axis.

### FIELD OF THE INVENTION

The present invention relates generally to measurement devices, methods, and documentation means. The invention relates specifically to an anatomic measurement device, method, and documentation means for medical assessment of disease states.

### BACKGROUND OF THE INVENTION

Anatomic measurement devices and methods are well known and have been in wide use by medical practitioners for, inter alia, assessing disease states or even for determining basic patient parameters in eventually making a detailed diagnosis. Examples of anatomic measurement devices could include rulers, calipers, scales, and, in general, devices that are capable of being placed against or near a patient's body to effect a desired measurement. Methods associated with such measurement devices are often readily apparent from the construction of the devices themselves, for example: placing a ruler between a patient's head and feet to determine the patient's height, manipulating a caliper to surround a mass of tissue to ascertain its dimensions, or placing the patient on a scale to note the patient's weight. Associated with these measurements have been relatively simple documentation means to record the findings, such as writing them on paper or typing them into computerized file programs.

Several difficulties could be encountered with use of known anatomic measurement devices for measurement of areas of interest in patients presenting unusual or uncommon anatomic disease states; and known documentation means have been inadequate for easily recording such measurements. For example, in Peyronie's Disease, a male penis acquires an abnormal curvature which is most pronounced when erect. The abnormal curvature is typically caused by scarring of internal penile tissue, that inhibits straightening of the penis and is most evident during an erection. In an extreme case of Peyronie's Disease, the curvature may be so pronounced that vaginal intercourse is nearly impossible. Treatment of Peyronie's Disease may be effected by orally-administered or intravenously-injected medicines, or by surgery when severe pain, significant penis curvature, or significant sexual dysfunction are indicated. In some cases, surgery may involve—either singularly or in combination—removal of scar tissue and shortening (plication) of an unaffected side of the penis. In other cases, a penile prosthetic device may be implanted or otherwise employed to maintain a more normal erection during intercourse. Regardless of a selected treatment method, however, it is usually necessary or at least desirable for a treating physician to perform a medical history or physical examination when Peyronie's Disease is indicated. In such examinations, various diagnostic and documentation methods have been employed ranging from simply taking a photograph of the penis while it is erect, to taking a radiographic (x-ray) image of the penis to ascertain its internal structure and whether any discernible scarring is present internally. Additionally, some physicians order a Doppler flow analysis to monitor blood flows in the penis between flaccid and erect states.

However, several difficulties could be encountered with use of these known anatomic measurement devices for measurement of areas of interest in patients presenting unusual or uncommon anatomic disease states such as, for example, the aforedescribed Peyronie's Disease; and known documentation means have been inadequate for easily recording such measurements. Chief among these difficulties are (i) a general inability to easily, quickly, inexpensively, formally, and repeatably measure areas of anatomical interest, and (ii) inconsistent or incomplete documentation of such measurements in patient files. For example, some physicians take photographs of areas of anatomical interest and simply place the photos in patient files for future reference. Clearly, these ad hoc photographic means, of varying quality and detail, do not provide any standardized, formal, or repeatable measurements or complete documentation thereof.

Thus, there exists a need for an anatomic measurement device, method, and documentation means for medical assessment of disease states that may overcome deficiencies of known devices, methods, and means. In particular, such devices, methods, and documentation means may provide, in particular, a way for physicians to easily, quickly, inexpensively, formally, and repeatably measure areas of anatomical interest, and to consistently and completely document such measurements.

Also, it is to be appreciated that novel aspects of the present invention—as will be understood by those in the medical arts—could be capable of use in and beneficial to virtually any practice wherein anatomic measurement devices, methods, and documentation means for medical assessment of disease states are desired. Such uses and benefits of the novel aspects of the present invention could be in, for example, diagnostic assessments and measurements of individual appendages such as, for example, fingers and toes of female and male professional athletes alike. But regardless of a particular embodiment, it is to be particularly understood that the novel aspects of the present invention could be advantageously employed in a wide variety of situations where such measurements or documentation are desired.

### SUMMARY OF THE INVENTION

In accordance with basic aspects of the present invention, an anatomic measurement device comprises a base sheet having a central longitudinal axis, a proximal base edge, a left side angle axis, and a right side angle axis. Grid markings are provided on the base sheet corresponding to the central longitudinal axis, the left side angle axis, and the right side angle axis.

Also in accordance with basic aspects of the present invention, a method of use of an anatomic measurement device comprises steps of (a) providing an anatomic measurement device having a base sheet with a central longitudinal axis, a proximal base edge, a left side angle axis, a right side angle axis, and grid markings on the base sheet, (b) placing an appendage on the anatomic measurement device with a base of the appendage positioned near the proximal base edge, (c) noting a length of the appendage with respect to the grid markings corresponding to the central longitudinal axis, and (d) noting a position of the appendage with respect to the grid markings corresponding to the left side angle axis and the right side angle axis.

Additionally in accordance-with basic aspects of the present invention, a method of manufacturing an anatomic measurement device comprises steps of (a) providing a base sheet having a central longitudinal axis, a proximal base edge, a distal base edge, a left side angle axis, a left base edge, a right side angle axis, and a right base edge, and (b) making a plurality of grid markings on the base sheet corresponding to the central longitudinal axis, the left side angle axis, and the right side angle axis.

Further in accordance with the present invention, a combined anatomic measurement device and anatomic measurement documentation means comprises a base sheet having a central longitudinal axis, a proximal base edge, a left side angle axis, and a right side angle axis. Grid markings are provided on the base sheet corresponding to the central longitudinal axis, the left side angle axis, and the right side angle axis A documentation portion is provided on the base sheet near the proximal base edge and outside of the grid markings.

Yet further in accordance with the present invention, a method of use of a combined anatomic measurement device and anatomic measurement documentation means comprises steps of (a) providing a combined anatomic measurement device and anatomic measurement documentation means having a base sheet with a central longitudinal axis, a proximal base edge, a left side angle axis, a right side angle axis, grid markings on the base sheet, and a documentation portion on the base sheet near the proximal base edge and outside of the grid markings, (b) placing an appendage on the combined anatomic measurement device and anatomic measurement documentation means, with a base of the appendage positioned near the proximal base edge, (c) noting a length of the appendage with respect to the grid markings corresponding to the central longitudinal axis, and (d) noting a position of the appendage with respect to the grid markings corresponding to the left side angle axis and the right side angle axis.

Still further in accordance with the present invention, a method of manufacturing a combined anatomic measurement device and anatomic measurement documentation means comprises steps of (a) providing a base sheet having a central longitudinal axis, a proximal base edge, a distal base edge, a left side angle axis, a left base edge, a right side angle axis, and a right base edge, (b) making a plurality of grid markings on the base sheet corresponding to the central longitudinal axis, the left side angle axis, and the right side angle axis, and (c) making a documentation portion on the base sheet near the proximal base edge and outside of the grid markings.

### BRIEF DESCRIPTION OF THE DRAWINGS

FIG. 1 is an example of an anatomic measurement device of the present invention.

FIG. 2 is an example of a combined anatomic measurement device and anatomic measurement documentation means, of the present invention.

### DETAILED DESCRIPTION OF THE INVENTION

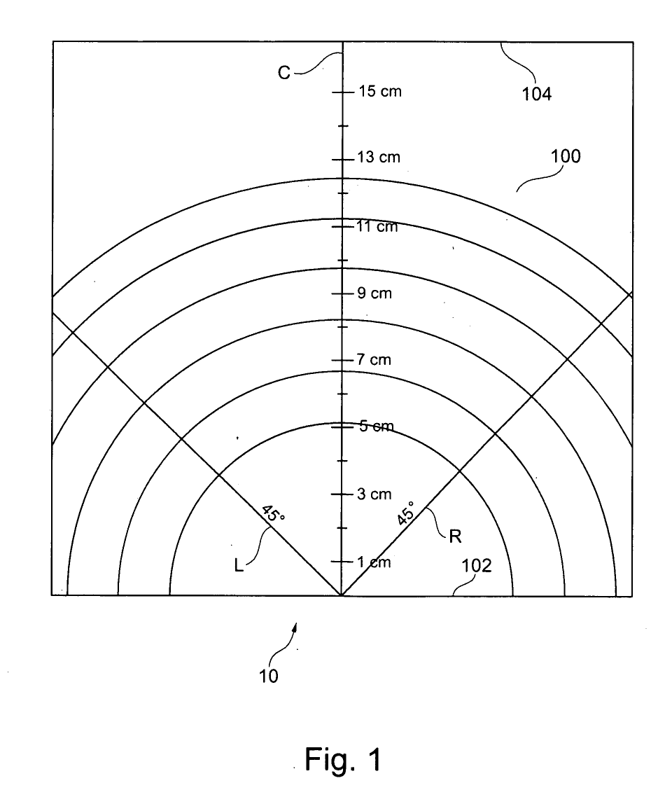

Illustrated in FIG. 1 is an example of an anatomic measurement device 10 (hereinafter, “device 10”). In this example, device 10 could include a base sheet 100 having a central longitudinal axis C, a proximal base edge 102, a distal base edge 104, a left side angle axis L, and a right side angle axis R. A plurality of grid markings (e.g., as shown in FIG. 1, in CM and degrees) could be provided on base sheet 100; and a purpose of such markings will be understood in a description below of use of device 10. These grid markings could correspond relatively to axes C, L, and R.

Upon suspecting or making an initial diagnosis of an unusual or uncommon anatomic disease state in a patient such as, for example, the aforedescribed Peyronie's Disease, a physician could utilize device 10 in the following manner. First, a flaccid penis (not illustrated) could be placed on base sheet 100 of device 10, with a base of the flaccid penis positioned near edge 102 of device 10. The physician could then note a length of the flaccid penis with respect to the grid markings (in CM) on base sheet 100 with respect to axis C. The physician could then cause the penis to become erect by any suitable treatment such as, for example, by an intravenous injection of chemicals into the corpus of the penis. The resulting erect penis (again, not illustrated) could be placed on base sheet 100 of device 10, with a base of the erect penis positioned near edge 102 of device 10. The physician could then note a length and angle of the erect penis with respect to the grid markings in CM and degrees on base sheet 100 relative to axes C, L and R.

It is to be appreciated that, although not illustrated, several alternatives could be employed in use of device 10 in the steps of noting a length of the flaccid penis and noting a position of the erect penis such as, for example: observing and recording measurements from device 10; taking a photograph of device 10 with the penis remaining thereupon; and drawing an outline of a selected portion of the penis on base sheet 100.

Device 10 could, advantageously and relatively inexpensively, be manufactured by way of a pad of printed sheets 100. Sheets 100 could be manufactured or printed to contain, for example, any of the aforedescribed grid markings as desired by a particular physician.

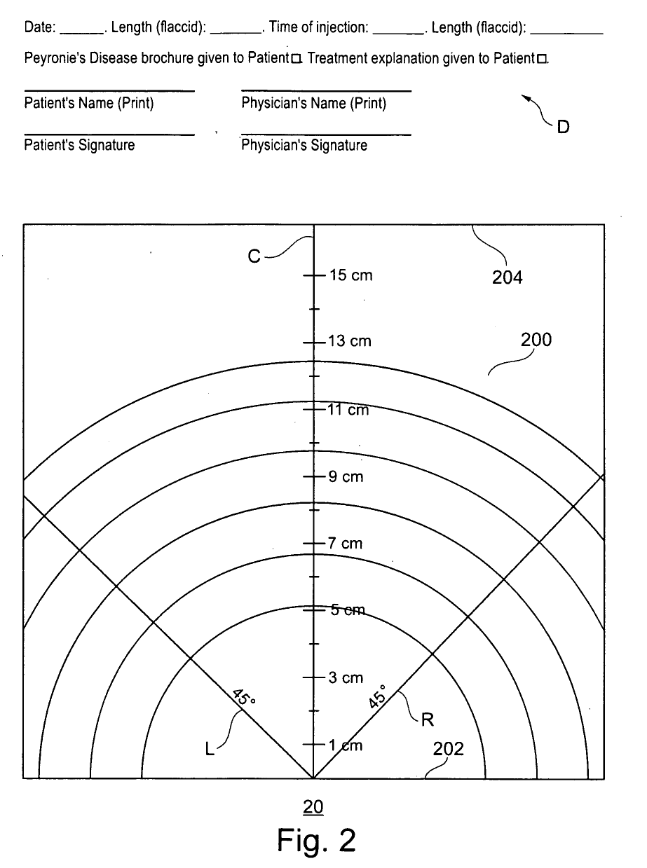

Illustrated in FIG. 2 is an example of a combined anatomic measurement device and anatomic measurement documentation means 20 (hereinafter, “device 20”). Similarities between combined device 20 the aforedescribed device 10 will be apparent from comparison of the two drawings, wherein similar reference numerals have been employed to describe similar elements of the invention. Most notably in FIG. 2, a documentation portion D could be provided directly on base sheet 200 near distal base edge 204, preferably outside of the grid markings.

Regardless of a particular configuration of devices of the present invention, it is to be understood that either example devices 10 or 20 could be physically stored in the patient's file for reference during any post-treatment appointments.

It is also to be appreciated from the foregoing disclosure that the present invention uniquely and advantageously satisfies the long-felt need for an anatomic measurement device, and for a combined anatomic measurement device and anatomic measurement documentation means, which allows physicians to (i) easily and repeatably measure areas of anatomical interest and (ii) consistently and completely document such measurements. Those of skill in the medical arts will appreciate that example device 10, and in particular example device 20, could effectively provide a “one-stop shop” for documenting anatomical measurements and for obtaining the patient's acknowledgment—as evidenced by the patient's signature—of receipt of information pertaining to, for example: indications and contraindications (via, perhaps, a brochure or patient literature); an explanation of proposed treatment; and verification of anatomical dimensions. In like manner, the physician could also document pre- and post-treatment indications and contraindications. Thus, clearly, example devices 10 and 20 could readily provide confirmation by the patient and file documentation by the physician for such purposes as “informed consent”, recording outcomes of treatment, and verification of what information was delivered to the patient—all in a standardized and uniform format.

Also, and as aforementioned, it is to be particularly appreciated that novel aspects of the present invention could be capable of use in assessments and measurements of individual appendages such as, for example, fingers and toes of female and male professional athletes alike. But regardless of a particular embodiment, it is to be particularly understood that the novel aspects of the present invention could be advantageously employed in any setting wherein anatomic measurement devices, methods, and documentation means for medical assessment of disease states are desired. Thus, the aforedescribed example of use of the present invention with respect to Peyronie's Disease could have instead been, for example, use of the present invention with respect to assessment, diagnosis, and treatment of the dislocated and often aggravated injury of a fifth finger (also termed “little finger” or “pinkie finger”) of professional basketball player Kobe Bryant of the Los Angeles Lakers franchise in the United States.

While the present invention has been particularly shown and described with reference to the accompanying specification and drawings, it will be understood however that other modifications thereto are of course possible; and all of which are intended to be within the true spirit and scope of the present invention. It should be appreciated that (i) components, dimensions, shapes, and other particulars of example embodiments of the invention aforedescribed may be substituted for others that are suitable for achieving desired results, (ii) various additions or deletions may be made thereto, and (iii) features of the foregoing examples may also be made in combinations thereof.

Lastly, of course, the choice of compositions, sizes, and strengths of various aforementioned elements of the present invention are all a matter of design choice depending upon intended uses thereof.

Accordingly, these and other various changes or modifications in form and detail of the present invention may also be made therein, again without departing from the true spirit and scope of the invention as defined by the appended claims.

What is claimed is:

1. An anatomic measurement device comprising:
a base sheet having a central longitudinal axis, a proximal base edge, a left side angle axis, and a right side angle axis; and
a plurality of grid markings on said base sheet, corresponding to (i) said central longitudinal axis, (ii) said left side angle axis, and (iii) said right side angle axis.

2. A method of use of an anatomic measurement device, comprising steps of:
providing an anatomic measurement device of claim 1;
placing an appendage on said anatomic measurement device with a base of the appendage positioned near said proximal base edge;
noting a length of the appendage with respect to said plurality of grid markings on said base sheet corresponding to said central longitudinal axis; and
noting a position of the appendage with respect to said plurality of grid markings on said base sheet corresponding to said left side angle axis and said right side angle axis.

3. The method of use of an anatomic measurement device of claim 2, wherein said steps of noting a length of the appendage and noting a position of the appendage are selected from a group consisting of: observing and recording measurements from said anatomic measurement device; taking a photograph of said anatomic measurement device; and drawing an outline of the appendage on said base sheet.

4. The method of use of an anatomic measurement device with respect to Peyronie's Disease, comprising steps of:
providing an anatomic measurement device of claim 1;
placing a flaccid penis on said anatomic measurement device with a base of the flaccid penis positioned near said proximal base edge;
noting a length of the flaccid penis with respect to said plurality of grid markings on said base sheet corresponding to said central longitudinal axis;
causing the flaccid penis to become an erect penis;
placing the erect penis on said anatomic measurement device with a base of the erect penis positioned near said proximal base edge; and
noting a position of the erect penis with respect to said plurality of grid markings on said base sheet corresponding to said left side angle axis and said right side angle axis.

5. The method of use of an anatomic measurement device with respect to Peyronie's Disease of claim 4, wherein said steps of noting a length of the flaccid penis and noting a position of the erect penis are selected from a group consisting of: observing and recording measurements from said anatomic measurement device; taking a photograph of said anatomic measurement device; and drawing an outline of the penis on said base sheet.

6. A method of manufacturing an anatomic measurement device of claim 1, comprising steps of:
providing a base sheet having a central longitudinal axis, a proximal base edge, a distal base edge, a left side angle axis, a left base edge, a right side angle axis, and a right base edge; and
making a plurality of grid markings on said base sheet corresponding to (i) said central longitudinal axis, (ii) said left side angle axis, and (iii) said right side angle axis.

7. A combined anatomic measurement device and anatomic measurement documentation means, comprising:
a base sheet having a central longitudinal axis, a proximal base edge, a left side angle axis, and a right side angle axis;
a plurality of grid markings on said base sheet, corresponding to (i) said central longitudinal axis, (ii) said left side angle axis, and (iii) said right side angle axis; and
a documentation portion on said base sheet near said proximal base edge and outside of said grid markings.

8. A method of use of a combined anatomic measurement device and anatomic measurement documentation means, comprising steps of:
providing a combined anatomic measurement device and anatomic measurement documentation means of claim 7;
placing an appendage on said combined anatomic measurement device and anatomic measurement documentation means, with a base of the appendage positioned near said proximal base edge;
noting a length of the appendage with respect to said plurality of grid markings on said base sheet corresponding to said central longitudinal axis; and
noting a position of the appendage with respect to said plurality of grid markings on said base sheet corresponding to said left side angle axis and said right side angle axis.

9. The method of use of a combined anatomic measurement device and anatomic measurement documentation means of claim 8, wherein said steps of noting a length of the appendage and noting a position of the appendage are selected from a group consisting of: observing and recording measurements from said anatomic measurement device; taking a photograph of said anatomic measurement device; and drawing an outline of the appendage on said base sheet.

10. The method of use of a combined anatomic measurement device and anatomic measurement documentation means with respect to Peyronie's Disease, comprising steps of:
providing a combined anatomic measurement device and anatomic measurement documentation means of claim 7;
placing a flaccid penis on said combined anatomic measurement device and anatomic measurement documentation means, with a base of the flaccid penis positioned near said proximal base edge;
noting a length of the flaccid penis with respect to said plurality of grid markings on said base sheet corresponding to said central longitudinal axis;
causing the flaccid penis to become an erect penis;
placing the erect penis on said combined anatomic measurement device and anatomic measurement documentation means with a base of the erect penis positioned near said proximal base edge; and
noting a position of the erect penis with respect to said plurality of grid markings on said base sheet corresponding to said left side angle axis and said right side angle axis.

11. The method of use of a combined anatomic measurement device and anatomic measurement documentation means with respect to Peyronie's Disease of claim 10, wherein said steps of noting a length of the flaccid penis and noting a position of the erect penis are selected from a group consisting of: observing and recording measurements from said anatomic measurement device; taking a photograph of said anatomic measurement device; and drawing an outline of the penis on said base sheet.

12. A method of manufacturing a combined anatomic measurement device and anatomic measurement documentation means of claim 7, comprising steps of:
providing a base sheet having a central longitudinal axis, a proximal base edge, a distal base edge, a left side angle axis, a left base edge, a right side angle axis, and a right base edge; and
making a plurality of grid markings on said base sheet corresponding to (i) said central longitudinal axis, (ii) said left side angle axis, and (iii) said right side angle axis; and
making a documentation portion on said base sheet near said proximal base edge and outside of said grid markings.
